(19) United States
(12) Patent Application Publication
(10) Pub. No.: US 2011/0269203 A1
(43) Pub. Date: Nov. 3, 2011

(54) Lipogenic zooplankton for the conversion of cellulosic biomass to biofuel

(76) Inventor: Randall Owen Sigle, Seattle, WA (US)

(21) Appl. No.: 12/770,639

(22) Filed: Apr. 29, 2010

(51) Int. Cl. C12P 5/00

(52) U.S. Cl. 435/166

(57) ABSTRACT

The present invention to provides a method for the conversion of biomass comprising cellulosic or carbohydrate polymers, into lipids useful for the production of biofuels. The method is achieved by providing the biomass to a culture of lipogenic zooplankton in a contained space, whereby the zooplankton consume the biomass, thereby converting the biomass to zooplankton derived biomass with increased lipid content. The zooplankton derived biomass is collected, lipids are extracted, and the lipids are converted to fuel through methods known in the art.

### TECHNICAL FIELD OF THE INVENTION

The present invention relates to biofuel production in general, and in particular the bioconversion of cellulosic biomass to hydrocarbons suitable for biofuel production.

### BACKGROUND OF THE INVENTION

Currently the majority of biofuel production relies on diversion of potential food crops to serve as the starting material for fuel production, such as corn for ethanol and oilseed crops for biodiesel. Such diversion increases demand on terrestrial agricultural resources and increases food prices worldwide. Cellulosic biomass is a promising alternative source of feedstock for fuel production, utilizing waste, marginal land, or even marine sources of biomass to decrease the demand on arable land. However, development of efficient processes for the conversion of cellulosic material to fuel remains a problem. Significant attention has been focused on the development of lipogenic algae for biofuels, but costs of production remain a barrier to cost-effectiveness.

The present invention addresses this problem by proposing an alternative method for the conversion of readily available cellulosic biomass to hydrocarbons utilizing the lipogenic capabilities of marine or freshwater zooplankton, particularly zooplankton of the subclass copepoda (copepods). Copepods are small crustaceans ranging in adult size from about a millimeter to about a centimeter in length. Copepod species are highly diverse, examples of which can be found occupying virtually any biome one earth. Copepods dominate zooplankton communities, are responsible for the majority of secondary biomass production in the oceans, and have been estimated to account for the majority of non-plant biomass in the ocean (Wikipedia:copepod). Certain copepod species, especially those found in extreme northern and southern latitudes, have been noted for their ability to synthesize and store lipids during times of abundant food supply which can be utilized during times of scarce food supply, or transferred to eggs to ensure egg survival. The lipid content of copepod adults or eggs has been observed to represent over 75% of the dry mass (Lee et al. 2006). Copepods can be carnivorous, omnivorous, herbivorous, or detrivorous. Harpacticoid copepods are meiobenthic, in that they attach themselves to suspended or settled detritus while they feed. This attribute which allows them to take advantage of the extensive surface area provided by the suspended particles, makes harpacticoid copepods particularly suited for intensive culture applications. Additionally, harpacticoids may be able to devote a higher proportion of their diet to lipid stores due to the energy not expended on swimming (Strottrup et al. 1997). Several intensive copepod culture systems have been developed mainly for the purpose of providing superior feed for larval fish. Production levels of 1.25 g dry copepod biomass per square meter per day have been achieved on a continuing basis (Sun et al. 1995). Scaleup of this production, assuming a 50% by dry weight lipid yield, could produce roughly 2000 Kg, or roughly 2000 liters of lipid per hectare per year, which compares quite favorably with the estimated yield of biodiesel from soybeans of 544 liters per hectare per year (Hill et al. 2006). In culture, harpacticoid copepods have been sustained on diets including cultivated algae, lettuce, frozen spinach, rice bran, commercial fish food flakes, and mixtures of tomato juice, brewer's yeast, vitamins and flax seed oil (Chandler 1986, Fraser et al. 1989, Kahan et al. 1982, Sun et al. 1995, Lemus et al. 2004, Rhodes 2003, Strottrup et al. 1997, Stottrup 2000). In the wild, harpacticoid copepods have been observed feeding in high densities on marine algae comprising Sargassum species Mukai 1971, and on fresh water algae as well (Caramujo et al. 2005). This invention represents a contribution to the prior art because lipogenic zooplankton so far have not been proposed as a lipid source for biofuels, but rather have been considered a nuisance parasite in algal cultures due to their propensity to consume such cultures (http://news.nationalgeographic.com/news/2009/03/090327-iron-seeding.html).

### SUMMARY OF THE INVENTION

It is the aim of the present invention to provide a method for the conversion of biomass comprising cellulosic or carbohydrate polymers, into lipids useful for the production of biofuels. The method is achieved by providing the biomass to a culture of lipogenic zooplankton in a contained space, whereby the zooplankton consume the biomass, thereby converting the biomass to zooplankton derived biomass with increased lipid content. The zooplankton derived biomass is collected, lipids are extracted, and the lipids are converted to fuel through methods known in the art.

### DETAILED DESCRIPTION OF THE INVENTION

The present invention anticipates the conversion of any biomass that can be consumed by zooplankton and converted into lipid containing zooplankton derived biomass (ZDB) utilizing the metabolic pathways of the zooplankton. The metabolic pathways comprise both de novo lipid synthesis and assimilation of lipids or lipid precursors provided in the diet. In a preferred embodiment, the biomass is Sargassum seaweed harvested from the Sargasso Sea as detailed in U.S. Pat. No. 7,479,167 B2 to Markels Jr. hereby incorporated by reference. The biomass may also comprise any other algae, such as naturally occurring marine or freshwater algae, algae that occurs as an unwanted invasive species, or algae that has been purposely cultivated. The biomass may comprise phytoplankton, diatoms, microalgae, or other organisms that may be consumed by the zooplankton. The biomass may comprise low value materials derived from terrestrial sources such as agricultural plant waste, animal waste, or municipal sewage.

The lipogenic zooplankton of the present invention comprise non-photosynthetic water-dwelling arthropods with the ability to synthesize lipids from metabolic precursors. The lipids may comprise wax esters, triacylglycerols, phospholipids, or alkyldiacylglycerol ethers as described (Lee et al. 2006). In a preferred embodiment, the zooplankton are harpacticoid copepods selected and isolated for their ability to accumulate high proportions of lipids into ZDB utilizing the provided biomass as a food source. The zooplankton may be obtained from naturally occurring populations or from isolated culture collections maintained in laboratory or hatchery conditions. The zooplankton may be primarily a single species or population of species selected to optimize the conversion of the biomass to lipid containing ZDB.

The zooplankton derived biomass (ZDB) may be any biomass that results from the metabolic activity of the zooplankton, comprising any stage of the life-cycle of the zooplankton, such as eggs, nauplii, intermediate stage copepodites, adults, and diapausing adults, or zooplankton waste such as carcasses or fecal pellets.

The zooplankton culture comprises the zooplankton selected for consumption of the biomass, and the biomass prepared in a manner suitable for consumption by the zooplankton, in a contained environment suitable for the maintenance of the metabolic activities of the zooplankton. The biomass may be provided in the form in which it is received or processed to enhance the ability of the zooplankton to consume it. Such processing steps may include mechanical processing such as chopping, grinding or pulverizing, chemical treatments, or enzymatic treatments. Additives may be used to enhance or maintain the metabolic activity of the culture. Additives may include nutritional supplements such as vitamins or essential fatty acids, feeding stimulants, or antibiotics to inhibit growth of undesired organisms. The containment may comprise a manmade enclosure, a natural enclosure, or a combination. Examples of manmade enclosures include artificial ponds, bioreactors, tanks, trays, bags, the hold of a ship or barge, or a floating pen or bag configured to contain the culture. Natural enclosures include ponds, lakes, bays, inlets, or ocean gyres (as described in U.S. Pat. No. 7,479,167 B2 to Markets Jr) that have the ability to confine the culture. An example of a combination manmade/natural enclosure is a bay or inlet provided with manmade bafflers that serve to confine the culture to the bay or inlet.

The present invention further comprises methods for the optimization of biomass conversion to lipid containing ZDB, including optimization of the zooplankton population for maximum lipid yield, and optimization of the culture conditions for maximum lipid yield. Optimization of culture conditions may include such parameters as the configuration of the enclosure, composition of the biomass, and physical parameters such as aeration, agitation, pH, culture density, temperature, freshwater exchange, and the like.

Methods for collection of lipids from zooplankton (Lee 2006), or U.S. Pat. No. 6,800,299 B1 to BEAUDOIN et al.), and conversion of biomass derived lipids into biofuel are well known in the art as detailed in U.S. Pat. No. 5,972,057 to Hayafuji et al. and US 2005/0112735 A1 to Zappi et al., each hereby incorporated by reference.

Other embodiments of the present invention may occur to one of skill in the art without departing from the scope or spirit of the invention.

### REFERENCES

Cited patent documents:

U.S. Pat. No. 7,479,167 B2 (MARKELS, Jr.) 20 Jan. 2009 (20.01.2009)

US 2005/0112735 A1 (ZAPPI et al.) 26 May 2005 (26.05.2005)

U.S. Pat. No. 5,972,057 A (HAYAFUJI et al.) 26 Oct. 1999 (26.10.1999)

U.S. Pat. No. 6,800,299 B1 (BEAUDOIN et al.) 05 Oct. 2004 (05.10.2004)

Non-Patent Literature:

Caramujo et al. 2005 Trophic interactions between benthic copepods and algal assemblages: a laboratory study J. N. Am. Benthol. Soc., 2005, 24(4):890-903

Chandler 1986 High-Density Culture of Meiobenthic Harpacticoid Copepods Within a Muddy Sediment Substrate Canadian Journal of Fisheries and Aquatic Sciences 43:53-59

Fraser et al. 1989 Lipid class and fatty acid composition of Calanus finmarchicus (Gunnerus), Pseudocalanus sp. And Temora longicomus Muller from a nutrient enriched seawater enclosure Journal of Experimental Marine Biology and Ecology 130:81-92

Hill et at. 2006 Environmental, economic, and energetic costs and benefits of biodiesel and ethanol biofuels 11206-11210_PNAS_Jul. 25, 2006_vol. 103_no. 30

Kahan et al. 1982 A Simple Method For Cultivating Harpacticoid Copepods And Offering Them To Fish Larvae Aquaculture 26:303-310

Sun et al. 1995 Sustained mass culture of Amphiascoides atopus a marine harpacticoid copepod in a recirculating system Aquaculture 136:313-321.

Lee et al. 2006 Lipid storage in marine zooplankton MARINE ECOLOGY PROGRESS SERIES 307:273-306.

Lemus et at. 2004 Increasing Production of Copepod Nauplii in a Brown-Water Zooplankton Culture with Supplemental Feeding and Increased Harvest Levels North American Journal of Aquaculture 66:169-176

Mukai 1971 The phytal animals on the thalli of Sargassum serratifolium in the Sargassum region, with reference to their seasonal fluctuations Marine Biology 8:170-182

Rhodes 2003 Methods for high density batch culture of Nitokra lacustris, a marine harpacticoid copepod The Big Fish Bang. Proceedings of the 26th Annual Larval Fish Conference. 2003. Edited by Howard I. Browman and Anne Befit Skiftesvik Published by the Institute of Marine Research, Postboks 1870 Nordnes, N-5817, Bergen, Norway. ISBN 82-7461-059-8

Strottrup et al. 1997 Production and use of Copepods in marine fish Iarviculture Aquaculture 155:231-247.

Stottrup 2000 The elusive copepods: their production and suitability in marine aquaculture Aquaculture Research 31:703-711

What is claimed is:

1. A method for conversion of cellulosic or carbohydrate biomass to hydrocarbon fuels comprising the steps of a).feeding the cellulosic or carbohydrate biomass to a contained culture of lipogenic zooplankton, wherein the zooplankton at least partially consume the biomass, and produce zooplankton derived biomass with an increased lipid content compared to the starting biomass; b). collecting the zooplankton derived biomass; c). purifying the lipids contained in the biomass produced by the zooplankton, and d).converting the lipids to a hydrocarbon fuel for combustion, wherein, the zooplankton derived biomass is used for the production of a hydrocarbon fuel

2. A method for adapting a population of zooplankton for the conversion of cellulosic or carbohydrate biomass to lipid containing zooplankton derived biomass comprising culturing the zooplankton in a medium containing cellulosic or carbohydrate biomass and selecting those zooplankton able to consume and convert the cellulosic or carbohydrate biomass into lipids contained in zooplankton derived biomass, wherein, the zooplankton derived biomass is used for the production of a hydrocarbon fuel, and wherein the selected population is more effective at producing lipids than the population prior to selection.

3. A method for the optimization of conversion of cellulosic or carbohydrate biomass into lipids suitable for the production of hydrocarbon fuel comprising culturing a population of zooplankton in a contained system with the cellulosic or carbohydrate biomass, and altering the containment system or culture conditions such that lipids are incorporated into zooplankton derived biomass, such that the yield of lipids in the zooplankton derived biomass is optimized, wherein, the zooplankton derived biomass is used for the production of a hydrocarbon fuel.

4. The method of any one of claims 1-3, wherein the zooplankton population comprises a harpacticoid copepod.

5. The method of any one of claims 1-3, wherein the biomass comprises algae of the genus Sargassum.

6. The method of any one of claims 1-3 wherein the biomass comprises algae of the genus Sargassum harvested from the Sargasso Sea.
